(19) United States
(12) Patent Application Publication
(10) Pub. No.: US 2025/0281478 A1
(43) Pub. Date: Sep. 11, 2025

(54) METHODS FOR PREVENTING OR TREATING KIDNEY DISEASES

(71) Applicant: JEAMOON BEIJING CO., LTD., Beijing (CN)

(72) Inventors: Qinghua CUI, Beijing (CN); Wanlu ZHOU, Beijing (CN)

(73) Assignee: JEAMOON BEIJING CO., LTD., Beijing (CN)

(21) Appl. No.: 19/215,282

(22) Filed: May 21, 2025

Related U.S. Application Data
(63) Continuation of application No. PCT/CN2023/110567, filed on Aug. 1, 2023.

(30) Foreign Application Priority Data
Nov. 22, 2022 (CN) 202211466949.1

(51) Int. Cl. A61K 31/4709; A61P 13/12

(52) U.S. Cl. CPC A61K 31/4709; A61P 13/12

(57) ABSTRACT

Provide is a method for preventing or treating a kidney disease. The method employs an adenine-induced mouse renal failure model to evaluate the therapeutic effects of LY2922470 and TAK875 on renal failure. The results demonstrate that LY2922470 has a therapeutic effect on renal failure, and this effect is not directly related to the GPR40 target. Additionally, an in vitro renal fibrosis model confirms that LY2922470 inhibits renal fibrosis. The significant potential of LY2922470 as a therapeutic agent for kidney diseases is verified. For the first time, it is revealed that LY2922470 effectively improves renal function, providing a promising novel candidate drug for the treatment of renal injury, chronic kidney disease, and renal failure. This expands the indications of LY2922470 and substantially enhances its application potential and market prospects.

### CROSS-REFERENCE TO RELATED APPLICATIONS

This application is a Continuation of International Patent Application No. PCT/CN2023/110567, filed on Aug. 1, 2023, which claims priority to Chinese Patent Application No. 202211466949.1, filed on Nov. 22, 2022, the entire content of each of which is hereby incorporated by reference.

### TECHNICAL FIELD

The present disclosure relates to the field of biomedicine, and particularly relates to a method for preventing or treating a kidney disease.

### BACKGROUND

LY2922470 was developed by Eli Lilly and Company as a GPR40 small-molecule activator for the treatment of type 2 diabetes mellitus (T2DM). LY2922470 has the following structural formula:

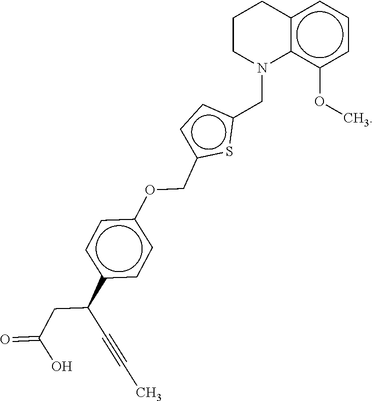

GPR40 belongs to the GPCR family. Activation of GPR40 has been demonstrated to induce glucose-stimulated insulin secretion (GSIS) in pancreatic B-cells and incretin secretion in enteroendocrine cells. As a result, GPR40 has emerged as a viable and promising therapeutic target for T2DM without the risk of hypoglycemia, garnering considerable attention as a drug target for T2DM. Numerous GPR40 ligands have been developed and studied for their antidiabetic effects. TAK875, a GPR40 activator, has been successfully tested in Phase II clinical trials.

The general structural formula of LY2922470 was first proposed in WO2015105786A1, which also indicated that this compound may be used to treat T2DM.

Failure of renal functions, also referred to as renal failure, is a syndrome characterized by a decline in glomerular filtration rate (GFR) and associated metabolic disorders and clinical symptoms caused by various chronic kidney diseases. It is marked by a sustained and progressive decrease in GFR. The incidence of chronic renal failure in China exceeds 5 per 10,000 of the total population. Apart from costly treatments such as hemodialysis and kidney transplantation, there is currently a lack of effective therapeutic drugs. Therefore, the search for new drugs to treat kidney diseases is of great significance.

Renal fibrosis is considered the common pathway of nearly all types of chronic kidney disease (CKD), affecting glomeruli, renal tubules, and renal blood vessels. Fibrosis is also a hallmark of CKD, characterized by epithelial-mesenchymal transition (EMT), excessive extracellular matrix (ECM) deposition, and the accumulation of interstitial and inflammatory cells. Through EMT, tubular epithelial cells transform into myofibroblasts, acquiring enhanced capabilities in cell proliferation, motility, contraction, and excessive ECM deposition, thereby leading to fibrosis. Injuries such as ischemia-reperfusion (IR), nephrotoxins, chemotherapeutic drugs, diabetes, hypertension, and ureteral obstruction can all contribute to the development of renal fibrosis.

Transforming growth factor-β1 (TGF-β1) exerts its effects by modulating the TGF-β/Smad pathway, making it a master regulator of fibrosis. Therefore, TGF-β1 may be used to induce an in vitro fibrosis model.

### SUMMARY

The technical problem addressed by the present disclosure is to provide a method for preventing or treating a kidney disease, comprising administering to a subject a medicament containing a pharmaceutically effective amount of LY2922470. As a drug for kidney diseases, LY2922470 can effectively improve renal function, offering a new alternative for the prevention and treatment of renal injury, chronic kidney disease, and renal failure.

One or more embodiments of the present disclosure provide a method for preventing or treating a kidney disease. The method comprises: administering to a subject a medicament containing a pharmaceutically effective amount of LY2922470. The LY2922470 has the following structural formula:

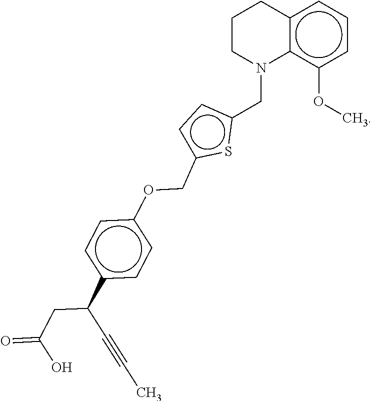

In some embodiments, the kidney disease is renal injury, chronic kidney disease, or renal failure.

In some embodiments, the kidney disease is nephropathy caused by renal fibrosis.

In some embodiments, the kidney disease includes at least one of ischemia-reperfusion (IR)-induced nephropathy, nephrotoxin-induced nephropathy, chemotherapeutic drug-induced nephropathy, hypertension-induced nephropathy, ureteral obstruction-induced nephropathy, and diabetic nephropathy.

In some embodiments, the LY2922470 in the medicament is in a form of a compound or a pharmaceutically acceptable salt thereof.

In some embodiments, the medicament is in an oral or injectable dosage form, and the oral or injectable dosage form includes at least one of a powder, a tablet, a granule, a capsule, an oral liquid, an emulsion, and a suspension.

In some embodiments, the medicament further includes an excipient or an adjunctive medicament for protecting kidneys and blood vessels.

In some embodiments, the medicament further includes a pharmaceutically acceptable carrier, the carrier includes at least one of a diluent, a buffer, a suspending agent, an emulsifying agent, a granulating agent, an encapsulating agent, an excipient, a filler, a binder, a spray agent, a transdermal absorption enhancer, a wetting agent, a disintegrant, an absorption enhancer, a surfactant, a colorant, a flavoring agent, and an adsorbent carrier.

### BRIEF DESCRIPTION OF THE DRAWINGS

FIG. 1 shows glomerular filtration rates in mice modeled by feeding with a 0.25% adenine-containing diet for three consecutive weeks;

FIG. 2 shows changes in serum creatinine (CREA) in an adenine-induced mouse model of renal failure (*P<0.05, **P<0.01, ***P<0.0001);

FIG. 3 shows survival curves of LY2922470-treated mice with adenine-induced renal failure;

FIG. 4 shows glomerular filtration rates in mice with sustained adenine-induced renal failure treated with LY2922470;

FIG. 5 shows glomerular filtration rate in mice with two-week adenine-induced renal failure treated with LY2922470;

FIG. 6 shows statistical analysis of renal fibrosis levels in LY2922470-treated mice with adenine-induced renal failure (semi-quantitative analysis of MASSON staining);

FIG. 7 shows RT-PCR quantitative results of fibrosis markers in TGF-β1-induced HK-2 cells treated with LY2922470; and

FIG. 8 shows RT-PCR quantitative results of fibrosis markers in high glucose-induced HK-2 cells treated with LY2922470.

### DETAILED DESCRIPTION

To provide a clearer illustration of the technical solutions in the embodiments of the present disclosure, the drawings required for describing the embodiments will be briefly introduced below. Obviously, the drawings in the following description are merely some examples or embodiments of the present disclosure. For those of ordinary skill in the art, other similar scenarios may be applied to the present disclosure based on these drawings without creative effort.

One or more embodiments of the present disclosure provide a method for preventing or treating a kidney disease. The method comprises: administering to a subject a medicament containing a pharmaceutically effective amount of LY2922470.

The LY2922470 has the following structural formula:

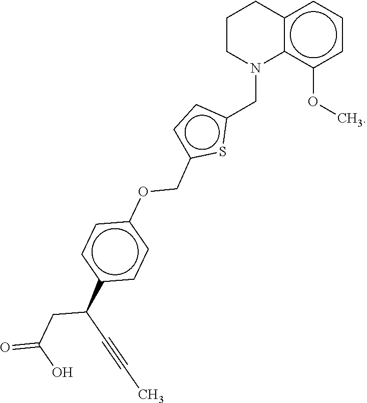

In some embodiments, the kidney disease is renal injury, chronic kidney disease, or renal failure.

In some embodiments, the kidney disease is nephropathy caused by renal fibrosis.

In some embodiments, the kidney disease includes at least one of ischemia-reperfusion (IR)-induced nephropathy, nephrotoxin-induced nephropathy, chemotherapeutic drug-induced nephropathy, hypertension-induced nephropathy, ureteral obstruction-induced nephropathy, and diabetic nephropathy.

In some embodiments, the LY2922470 in the medicament is in a form of a compound or a pharmaceutically acceptable salt thereof.

In some embodiments, the medicament is in an oral or injectable dosage form, and the oral or injectable dosage form includes at least one of a powder, a tablet, a granule, a capsule, an oral liquid, an emulsion, and a suspension.

In some embodiments, the medicament further includes an excipient or an adjunctive medicament for protecting kidneys and blood vessels.

In some embodiments, the medicament further includes a pharmaceutically acceptable carrier, the carrier includes at least one of a diluent, a buffer, a suspending agent, an emulsifying agent, a granulating agent, an encapsulating agent, an excipient, a filler, a binder, a spray agent, a transdermal absorption enhancer, a wetting agent, a disintegrant, an absorption enhancer, a surfactant, a colorant, a flavoring agent, and an adsorbent carrier.

One or more embodiments of the present disclosure provide a use of LY2922470 in the preparation of a medicament for preventing or treating a kidney disease, wherein the LY2922470 has the following structural formula:

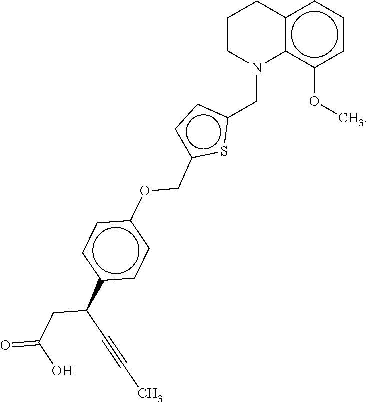

In some embodiments, the kidney disease is renal injury, chronic kidney disease, or renal failure.

In some embodiments, the kidney disease is nephropathy caused by renal fibrosis.

In some embodiments, the kidney disease includes at least one of ischemia-reperfusion (IR)-induced nephropathy, nephrotoxin-induced nephropathy, chemotherapeutic drug-induced nephropathy, hypertension-induced nephropathy, ureteral obstruction-induced nephropathy, and diabetic nephropathy.

In some embodiments, the LY2922470 in the medicament is in a form of a compound or a pharmaceutically acceptable salt thereof.

In some embodiments, the medicament is in an oral or injectable dosage form, and the oral or injectable dosage form includes at least one of a powder, a tablet, a granule, a capsule, an oral liquid, an emulsion, and a suspension.

In some embodiments, the medicament further includes an excipient or an adjunctive medicament for protecting kidneys and blood vessels.

In some embodiments, the medicament further includes a pharmaceutically acceptable carrier, the carrier includes at least one of a diluent, a buffer, a suspending agent, an emulsifying agent, a granulating agent, an encapsulating agent, an excipient, a filler, a binder, a spray agent, a transdermal absorption enhancer, a wetting agent, a disintegrant, an absorption enhancer, a surfactant, a colorant, a flavoring agent, and an adsorbent carrier.

In some embodiments of the present disclosure, the medicament contains a therapeutically effective amount of LY2922470. The therapeutically effective amount refers to an amount of an active ingredient per unit dosage form (e.g., one tablet, one injection, one pill, or one dose of the drug) or a unit dose per treated subject (e.g., dose per unit body weight). In the present disclosure, the treated subjects are mammals, including humans, canines, rodents, etc. Conversion of therapeutically effective amounts between different species may be based on established equivalent dose relationships between experimental animals and humans, as commonly referenced in guidelines from regulatory agencies such as the FDA or SFDA. Reference may also be made to “Huang Jihan, et al. Dose conversion between animals and humans in pharmacological studies. Chinese Journal of Clinical Pharmacology and Therapeutics, 2004 September; 9 (9): 1069-1072.” Based on such conversion relationships, the human dose per unit body weight may be derived from the dose used in experimental animals. For example, in the case of commonly used experimental animals such as mice, the conversion ratio to adult humans is approximately 12:1 according to the aforementioned literature.

In some embodiments, in 8-week-old C57BL/6N mice, the therapeutically effective amount for significantly treating adenine-induced renal failure is in a range from 20 mg/kg to 100 mg/kg (by content). Preferably, the therapeutically effective amount is in a range from 30 mg/kg to 60 mg/kg.

Preferably, based on the dose conversion relationship between mice and adult humans and assuming a standard adult body weight of 60 kg, the therapeutically effective amount for adults is in a range from 100 mg to 500 mg per day. More preferably, the therapeutically effective amount for adults is in a range from 150 mg to 300 mg per day.

The embodiments of the present disclosure provide at least the following beneficial effects:

By using an adenine-induced renal failure animal model to evaluate therapeutic effects of LY2922470 and TAK875 on renal injury and renal failure, the results showed that LY2922470 exerts a therapeutic effect on renal failure, and the effect is not directly related to a GPR40 target. In vitro renal fibrosis models confirmed that LY2922470 also has an inhibitory effect on renal fibrosis. The embodiments disclosed herein demonstrate the significant potential of LY2922470 as a therapeutic agent for kidney diseases, and for the first time, reveal that LY2922470 effectively improves renal function. This provides a novel and effective therapeutic candidate for the treatment of renal injury, chronic kidney disease, and renal failure, expands the indications of LY2922470, and greatly enhances its application potential and market prospects.

The following examples provide more specific descriptions related to the above-mentioned embodiments. Certain elements in these examples may also be replaced with or combined with corresponding elements in other examples to form new examples. Unless otherwise specified, the experimental techniques described in the following examples are conventional techniques. Unless otherwise specified, the experimental materials used in the following examples were obtained from routine biochemical reagent suppliers. For quantitative assays in the following examples, all experiments were conducted in triplicate, and the results are presented as the mean values. It should be understood that these examples are provided to further illustrate the embodiments of the present disclosure, and are not intended to limit the scope of the present disclosure.

Unless otherwise specified, the experimental techniques used in the following examples are standard protocols. Experimental materials employed, unless otherwise indicated, may be obtained through commercial sources.

### EXAMPLES

### Example 1: Establishment of a Mouse Model of Renal Injury and Renal Failure Induced by Adenine

### I. Experimental Materials

1. Experimental Animals: C57BL/6N mice, 8 weeks old, SPF grade, male, provided by SPF (Beijing) Biotechnology Co., Ltd.

### II. Modeling Approach for Mouse Renal Injury and Renal Failure (Adenine-Induced)

Except for the control group, the other mice were fed a 0.25% adenine-containing diet for modelling. The control group was fed a standard diet. All mice had free access to food. The modeling lasted for three weeks. At the end of the third week, glomerular filtration rate (GFR) and relevant renal function indicators were assessed.

### III. Experimental Results and Conclusion

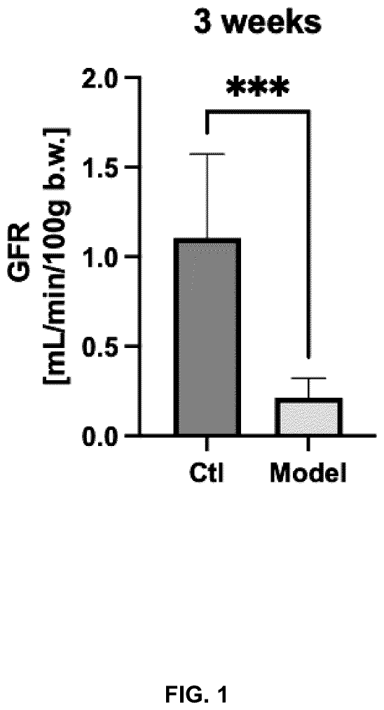

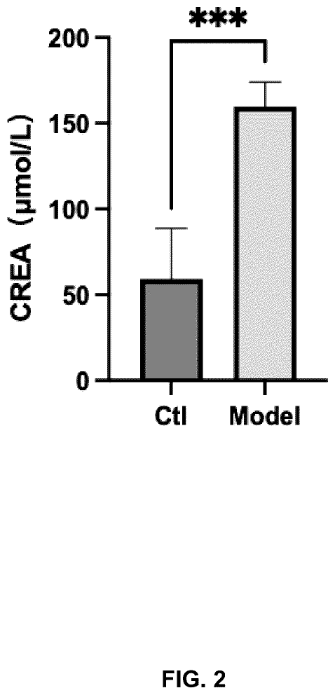

GFRs of the control group and the adenine-induced renal failure model group are shown in FIG. 1. Compared with mice fed a standard diet, the mice fed with the 0.25% adenine-containing diet showed a significant decrease in GFR (P<0.001), indicating a near-complete loss of glomerular filtration function. The concentration of serum creatinine (CREA) is primarily determined by a glomerular filtration capacity (e.g., the GFR). As the filtration capacity declines, creatinine concentration increases. Serum creatinine levels can accurately reflect renal parenchymal damage. CREA levels in the control and model groups are shown in FIG. 2. Compared with the control group, the CREA level in the model group was significantly elevated (P<0.001), consistent with the physiological and pathological characteristics of renal failure in mice, indicating successful establishment of a renal failure model.

### Example 2: LY2922470 Effectively Reduces Mortality in Mice with Renal Failure

### I. Experimental Materials Attorney Docket No. 61211-H002US00

1. Experimental Animals: C57BL/6N, 8 weeks old, SPF grade, male.
2. Experimental Drugs:
LY2922470 (Shanghai Taoshu Biotechnology Co., Ltd.)
TAK875 (Shanghai Taoshu Biotechnology Co., Ltd.)

1) Preparation of TAK875: A total of 27 mg of TAK875 powder was accurately weighed using an analytical balance and dissolved in 9 mL of 0.8% CMC-Na solution. The mixture was sonicated for 5 minutes to form a 3 mg/mL white suspension. The suspension was stored at 4° C. for later use.

2) Preparation of LY2922470: A total of 80 mg of LY2922470 yellow crystals was added to 200 μL of DMSO to form a clear yellow solution. The solution was gradually added in batches to 10 mL of 0.8% CMC-Na solution. After heating and sonication for 5 hours, an 8 mg/mL white suspension of LY2922470 was obtained. The suspension was stored at 4° C. for later use.

### II. Experimental Grouping and Drug Administration

| | Animal Count | | Administration |
|---|---|---|---|
| Group | (n) | Dose | Route |
| Control (Ctl) | 8 | — | p.o., QD |
| Model | 12 | — | p.o., QD |
| TAK875 | 12 | 30 mg/kg | p.o., QD |
| LY Low Dose | 12 | 10 mg/kg | p.o., QD |
| LY Medium Dose | 12 | 30 mg/kg | p.o., QD |
| LY High Dose | 12 | 60 mg/kg | p.o., QD |

Notes:
p.o.: per os (oral administration);
QD: once daily

### III. Experimental Procedure

Eight-week-old male C57BL/6N mice were acclimatized for one week and then randomly divided into six groups. Except for the control group, all mice were fed a 0.25% adenine-containing diet for modelling. The control group received a normal diet. The mice had free access to food. One week after model induction, mice in each group were orally administered the corresponding drug once daily in the afternoon, according to their group assignment. The control group and the model group received vehicle under a same condition. During the treatment period, the adenine-containing diet was continued. Mouse survival was monitored until the end of the experiment.

### IV. Experimental Results and Conclusion

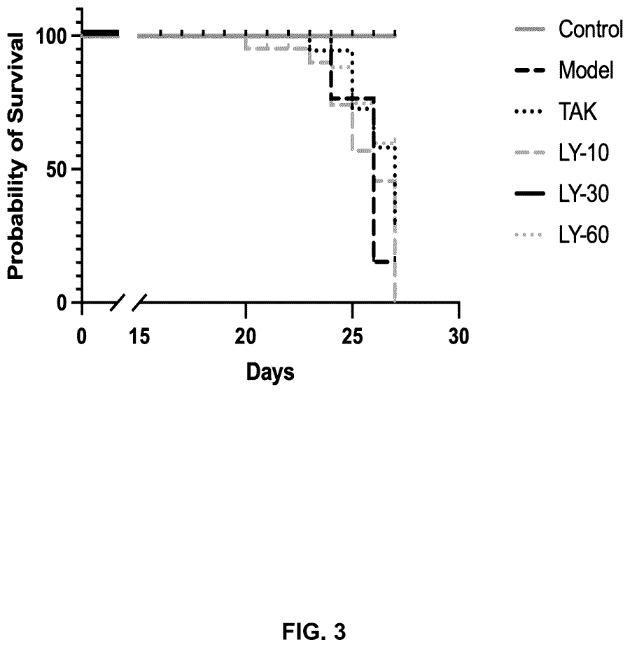

Table 1 presents survival rates of mice in the adenine-induced renal failure model treated with LY2922470, and FIG. 3 shows the corresponding survival curves. No mouse deaths occurred in any group within the first 19 days of adenine feeding, although weight loss and visible frailty were observed. Mouse deaths began on day 20 of adenine feeding, and by day 27, all mice in the LY-10 mg/kg group had died. The survival rate in the control group was 100%, while the model group showed a survival rate of only 14.3%. The TAK875 group had a survival rate of 46.2%. The LY-60 mg/kg group had a survival rate similar to the TAK875 group (50%). The strongest renal protective effect of LY2922470 was observed at 30 mg/kg, with a final survival rate of 100%, double that of the TAK875 group.

Data from the table indicate that LY2922470 exhibits therapeutic effects at doses ranging from 30 mg/kg/day to 60 mg/kg/day, with the optimal effect at 30 mg/kg/day. However, the potential renal protective effects of doses below 30 mg/kg/day (e.g., 20-30 mg/kg) and therapeutic effects of doses above 60 mg/kg/day (e.g., 60-100 mg/kg) may not be excluded.

TABLE 1
Survival Rates of Mice in the Adenine-Induced
Renal Failure Model Treated with LY2922470

| | | Count after | |
|---|---|---|---|
| | Count at | Drug | Survival Rate |
| Group | Enrollment | Administration | (%) |
| Control | 8 | 8 | 100% |
| Model | 14 | 2 | 14.30% |
| TAK875 | 13 | 6 | 46.20% |
| LY-10 mg/kg | 12 | 0 | 0% |
| LY-30 mg/kg | 12 | 12 | 100% |
| LY-60 mg/kg | 12 | 6 | 50% |

### Example 3: Therapeutic Effects of LY2922470 on Renal Injury and Renal Failure

### I. Experimental Materials

1. Experimental animals: C57BL/6N male mice, 8 weeks old, SPF grade.
2. Experimental drugs: LY2922470 and TAK875 (both purchased from Shanghai Taoshu Biotechnology Co., Ltd.).

### II. Experimental Grouping

a) Control group: Fed with adenine-free feed; other procedures were the same as those in the model group.
b) Model group: Fed with feed containing 0.25% adenine for modelling.
c) LY-10 mg/kg group: Model+LY2922470 (10 mg/kg, p.o., QD).
d) LY-30 mg/kg group: Model+LY2922470 (30 mg/kg, p.o., QD).
e) LY-60 mg/kg group: Model+LY2922470 (60 mg/kg, p.o., QD).
f) TAK875 group: Model+TAK875 (30 mg/kg, p.o., QD).

Notes: p.o.: per os (oral administration); QD: once daily.

### III. Experimental Technique

### 1. Experimental Procedure

Except for the control group, all other mice were fed with a diet containing 0.25% adenine for modelling. The control group was fed with a normal diet. All mice were allowed free access to food. One week after modeling, each group of mice was administered the respective treatment via oral gavage once daily in the afternoon for two consecutive weeks. The control group and the model group received vehicle under a same condition. The adenine-containing diet was continuously administered for modeling throughout the treatment period until the end of the experiment. The physical condition of the mice was monitored, and body weight was measured and recorded weekly. Blood samples were collected from the orbital venous plexus at 1 week (post-modeling) and 3 weeks (after 2 weeks of treatment). Serum was separated for the measurement of serum creatinine (CRE) and blood urea nitrogen (BUN). At the end of the two-week treatment, renal function of each group was assessed using a TGFR renal function monitoring and smart analysis system. After testing, tissues were collected from the mice to confirm the efficacy of the treatment.

### 2. Data Collection and Statistical Analysis

Statistical analyses were performed using GraphPad Prism 8.0 software. Continuous data of each group were expressed as mean+SD. One-way ANOVA was used for multi-group comparisons, and Student's t-test was used for comparisons between two groups. A p-value<0.05 was considered statistically significant. For categorical data, chi-square tests were used to compare between groups, with p<0.05 indicating a statistically significant difference.

### Experimental Results and Conclusions

### 1. Glomerular Filtration Rate (GFR)

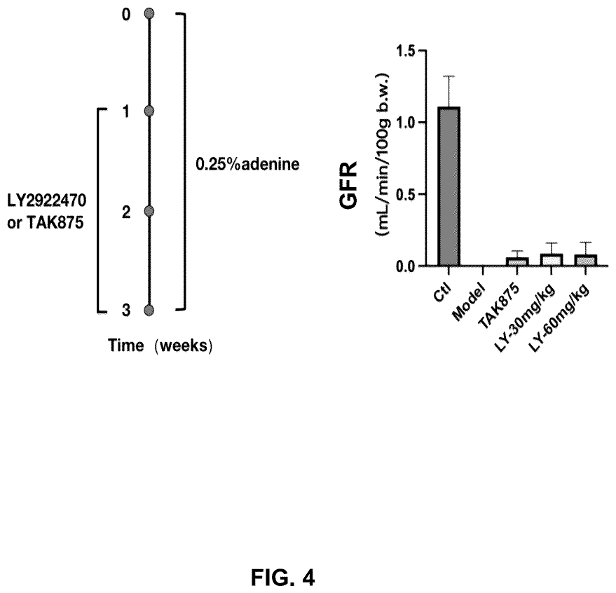

As shown in FIG. 4, mice were continuously fed a diet containing 0.25% adenine. One week later, LY2922470 or TAK875 was administrated for treatment. During the treatment period, the adenine-containing diet was continued to induce modeling. After three weeks of adenine feeding (i.e., two weeks of drug administration), the TGFR renal function monitoring and smart analysis system was used to evaluate the glomerular filtration rate in mice.

As shown in FIG. 4, compared to the control group, the model group showed a marked decrease in GFR, indicating near-complete loss of glomerular function. Compared with the model group, oral administration of TAK875, a GPR40 agonist, increased GFR in mice. Oral administration of LY2922470 at different doses also improved renal function in mice, with the most significant improvement observed at 30 mg/kg.

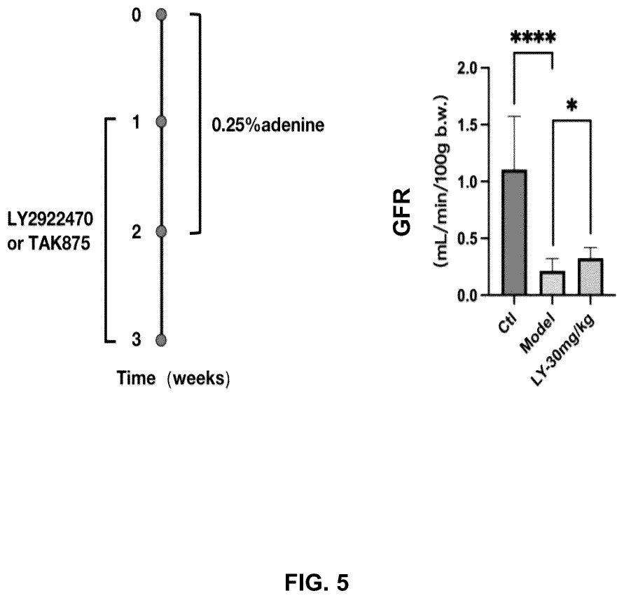

The above model induced by continuous feeding of 0.25% adenine resulted in excessive renal failure in mice, causing severe kidney damage. Therefore, the modeling protocol was redesigned to re-evaluate the therapeutic effect of LY2922470 on renal failure (the adenine diet was replaced with normal chow after two weeks of feeding; other conditions remained unchanged). The results are shown in FIG. 5. Compared with the model group, renal function in mice treated with LY2922470 was significantly improved, with statistical significance (NS: not significant, *p<0.5, **p<0.01, ***p<0.001). These results demonstrate that LY2922470 has therapeutic effects on renal injury and renal failure.

### 2. Renal Function Markers: Serum Creatinine (CREA) and Blood Urea Nitrogen (BUN)

Mice were fed a 0.25% adenine-containing diet. After one week, the mice were treated with LY2922470 or TAK875. During the treatment period, the adenine-containing diet was continued for modeling. After three weeks of adenine feeding (i.e., two weeks of drug administration), blood samples were collected from the mice, and serum was isolated for CREA and BUN measurements. The renal function indicators CREA and BUN for each group are shown in Tables 2 and 3.

After one week of adenine feeding, there was no significant difference in renal function among the groups. After three weeks of modeling (i.e., two weeks of drug administration), both CREA and BUN levels were elevated in the model group compared to the control group (#p<0.05, ##p<0.01). Compared with the model group, oral administration of the GPR40 agonist TAK875 did not significantly reduce CREA and BUN levels (*p<0.5, **p<0.01, ***p<0.001). However, oral administration of LY2922470 at different doses reduced serum CREA and BUN levels in mice. Compared with the model group, CREA levels significantly decreased in the LY-30 mg/kg and LY-60 mg/kg groups (i.e., mice treated with 30 mg/kg and 60 mg/kg LY2922470), and the LY-30 mg/kg group also showed a statistically significant reduction in BUN levels. These results indicate that LY2922470 has therapeutic effects on renal injury and renal failure, and that these effects are not directly related to the GPR40 target.

Based on the data in the tables, LY2922470 demonstrated therapeutic efficacy at doses of 30-60 mg/kg/day, with the best effect observed at 30 mg/kg/day. However, the potential renoprotective effects of doses lower than 30 mg/kg (e.g., 20-30 mg/kg) or higher doses (e.g., 60-100 mg/kg) may not be excluded.

TABLE 2
Serum CREA levels in each group of mice

CREA(μmol/L)

| | | After modeling for 3 |
|---|---|---|
| | After modeling for 1 | weeks (after drug |
| | week (before drug | administration for 2 |
| Group | administration) | weeks) |
| Control | 53.74 ± 14.79 | 59.25 ± 29.55 |
| Model | 53.87 ± 14.56 | 159.75 ± 14.42## |
| TAK | 46.54 ± 113.22 | 137.68 ± 30.23 |
| LY-10 mg/kg | 56.14 ± 13.41 | — |
| LY-30 mg/kg | 54.85 ± 17.55 | 79.67 ± 19.28** |
| LY-60 mg/kg | 57.02 ± 11.90 | 66.47 ± 13.35** |

TABLE 3
Serum BUN levels in each group of mice

BUN(mmol/L)

| | | After modeling for 3 |
|---|---|---|
| | After modeling for 1 | weeks (after drug |
| | week (before drug | administration for 2 |
| Group | administration) | weeks) |
| Control | 11.36 ± 3.64 | 12.70 ± 8.17 |
| Model | 10.63 ± 3.47 | 24.72 ± 11.83 |
| TAK | 11.25 ± 3.98 | 25.07 ± 4.91 |
| LY-10 mg/kg | 10.34 ± 3.55 | — |
| LY-30 mg/kg | 10.25 ± 3.42 | 15.15 ± 4.53* |
| LY-60 mg/kg | 10.40 ± 3.83 | 13.85 ± 1.33 |

### Example 4: Inhibitory Effect of LY2922470 on Renal Fibrosis

### I. Experimental Materials

Same as in Example 3.

### II. Experimental Grouping

Same as in Example 3.

### III. Experimental Techniques

### 1. Experimental Procedures

Mice were modeled and administered drugs as described in Example 3. After two weeks of drug administration, the mice were euthanized, and their kidneys were fixed, sectioned, stained with Masson's trichrome, and analyzed.

### 2. Data Collection and Statistical Analysis

Whole-slide scanning was performed using a fully automated digital pathology scanner KF-PRO-120 from Ningbo Jiangfeng Bioinformatics Technology Co., Ltd. Masson-stained images were analyzed using the Area Quantification module in HALO software. The blue staining of collagen fibers was used as the unified criterion for positivity. The images were analyzed, and the percentage of blue-stained collagen fibers in the tissue was calculated.

Statistical analysis was conducted using GraphPad Prism 8.0. Continuous data for each group were expressed as mean+SD. One-way ANOVA was used for multi-group comparisons, and Student's t-test was used for comparisons between two groups. A p-value<0.05 was considered statistically significant. For categorical data, chi-square tests were used for comparisons among groups, and a p-value<0.05 was considered statistically significant.

### IV. Experimental Results and Conclusion

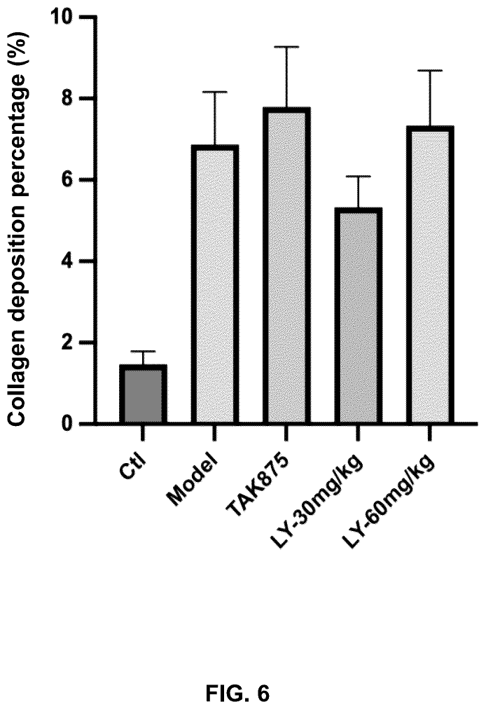

As shown in FIG. 6, after three weeks of modeling (i.e., two weeks of drug administration), a degree of renal fibrosis in the model group was significantly increased compared to the control group. Compared to the model group, the degree of fibrosis was not significantly reduced after oral administration of the GPR40 agonist TAK875. In mice orally administered 30 mg/kg/day LY2922470, collagen deposition decreased, extracellular matrix (ECM) accumulation was inhibited, and the degree of renal fibrosis was alleviated. The experimental results indicate that LY2922470 can inhibit renal fibrosis, and the anti-fibrotic effect may occur independently of the GPR40 target.

### Example 5: Inhibitory Effect of LY2922470 on Renal Fibrosis (TGF-β1-Induced Fibrosis)

### Experimental Materials

| Name | Manufacturer | Catalog No. |
|---|---|---|
| HK-2 cells | Procell | CL-0109 |
| Minimum Essential Medium | Procell | PM150410 |
| (MEM) | | |
| FBS | Lonsera | |
| Penicillin-Streptomycin Solution | Biosharp | BL505A |
| 0.25% Trypsin | CTCC | 002PI |
| Phosphate-buffered saline (PBS) | CTCC | 013PI |
| Other chemical reagents | Domestic analytical | |
| | grade | |

### II. Experimental Grouping

Group A: Control
Group B: TGF-β1 (10 ng/ml)
Group C: TGF-β1 (10 ng/ml)+LY (10 nM)
Group D: TGF-β1 (10 ng/ml)+LY (50 nM)
Group E: TGF-1 (10 ng/ml)+LY (500 nM)

### 3. Experimental Techniques

HK-2 cells were cultured in MEM supplemented with 10% FBS and 1% penicillin-streptomycin solution. The HK-2 cells were taken from liquid nitrogen and quickly thawed in a 37° C. water bath, gently agitated to thaw the cryopreservation medium. After thawing, the cell suspension was transferred to a centrifuge tube containing 3 mL of culture medium and centrifuged at 1000 rpm for 5 min at room temperature to collect the cells. The supernatant was discarded. The cell pellet was resuspended in complete medium (10% FBS) and seeded into culture dishes. The cells were gently pipetted to ensure uniform distribution and incubated at 37° C. with 5% CO₂ and saturated humidity.

HK-2 cells in a logarithmic growth phase and with good viability were seeded into 24-well plates at a density of 1×10⁵ cells/well. Once the cells reached 40-50% confluence, treatments were applied according to the grouping described above: Group A: HK-2 cells were cultured normally; Group B was treated with 10 ng/ml TGF-β1; Group C was treated with 10 ng/ml TGF-β1 and 10 nM LY2922470; Group D was treated with 10 ng/ml TGF-β1 and 50 nM LY2922470; and Group E was treated with 10 ng/ML TGF-β1 and 500 nM LY2922470. All groups were cultured for 48 hours before sample collection for RT-PCR analysis. Primer sequences are listed below:

| Primer | Sequence (5′-3′) |
|---|---|
| Fibronectin (human)-RT-F | GAAGTGGGACCGTCAGGGAGAAA |
| Fibronectin (human)-RT-R | CAGGAGCAAATGGCACCGAGATA |
| α-SMA (human)-RT-F | CCCCTAAACCCTAAAGCCAACA |
| α-SMA (human)-RT-R | GCAGTGCATAGCCCTCGTAGAT |
| Gapdh (human)-RT-E | GGAGCGAGATCCCTCCAAAAT |
| Gapdh (human)-RT-R | GGCTGTTGTCATACTTCTCATGG |

### 4. Experimental Results

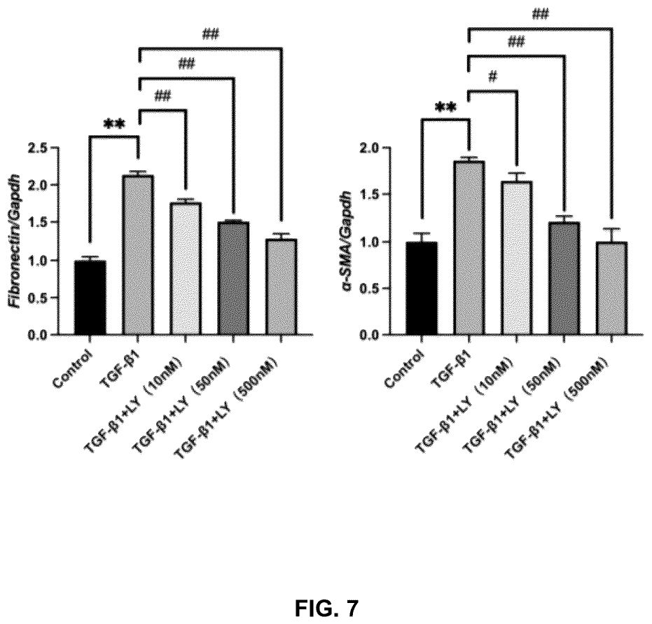

A TGF-1-induced human renal cortical proximal tubule epithelial cell (HK-2) model was used to confirm the anti-fibrotic effect of LY2922470. RT-PCR results (FIG. 7) showed that compared to the control group (Group A), treatment with TGF-β1 significantly increased the expression levels of fibrotic markers Fibronectin and α-SMA. Compared to the TGF-β1 group, treatment with varying concentrations of LY2922470 led to a dose-dependent decrease in the expression levels of Fibronectin and α-SMA (*: p<0.05 vs. Control; **: p<0.01 vs. Control; #: p<0.05 vs. TGF-β1 group; ##: p<0.01 vs. TGF-β1 group).

### Example 6: Inhibitory Effect of LY2922470 on Renal Fibrosis (High Glucose-Induced Fibrosis)

### I. Experimental Materials

1. D-glucose (Shanghai Pharmaceutical Group).
2. The remaining materials are the same as in Example 5.

### II. Experimental Grouping

Group A: Control
Group B: HG (25 mM)
Group C: HG (25 mM)+LY (10 nM)
Group D: HG (25 mM)+LY (50 nM)
Group E: HG (25 mM)+LY (500 nM)

### III. Experimental Techniques

HK-2 cells were cultured in MEM supplemented with 10% FBS and 1% penicillin-streptomycin solution. The HK-2 cells were taken from liquid nitrogen and quickly thawed in a 37° C. water bath, gently agitated to thaw the cryopreservation medium. After thawing, the cell suspension was transferred to a centrifuge tube containing 3 mL of culture medium and centrifuged at 1000 rpm for 5 min at room temperature to collect the cells. The supernatant was discarded. The cell pellet was resuspended in complete medium (10% FBS) and seeded into culture dishes. The cells were gently pipetted to ensure uniform distribution and incubated at 37° C. with 5% CO₂ and saturated humidity.

Once cells reached 40%-50% confluency, they were treated according to the grouping described above: Group A received normal HK-2 culture; Group B was treated with 25 mM D-glucose (high glucose); Group C was treated with 25 mM D-glucose and 10 nM LY2922470; Group D was treated with 25 mM D-glucose and 50 nM LY2922470; and Group E was treated with 25 mM D-glucose and 500 nM LY2922470. After 48 hours of culture, samples were collected from each group for RT-PCR analysis.

### IV. Experimental Results

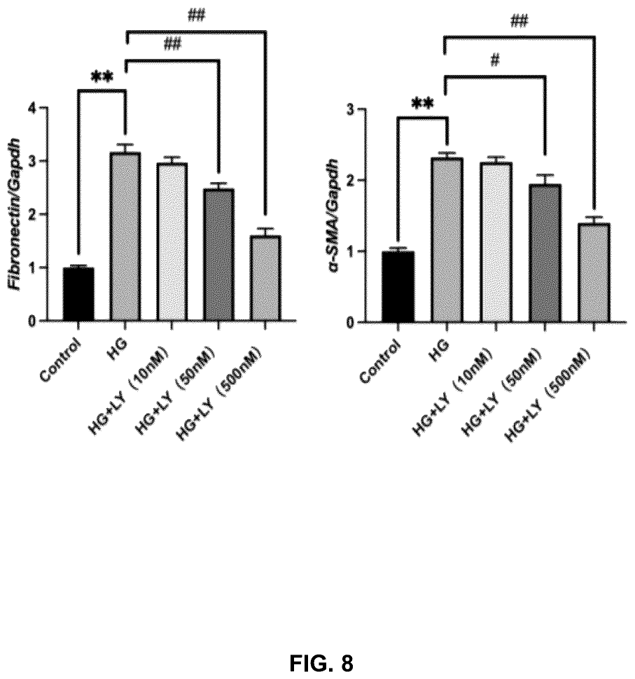

A high glucose-induced HK-2 injury model was used to confirm the anti-fibrotic effect of LY2922470. The RT-PCR results (FIG. 8) showed that compared to the control group, high-glucose treatment significantly increased the expression levels of fibrotic markers Fibronectin and α-SMA. Compared to the high-glucose group, treatment with various concentrations of LY2922470 reduced the expression levels of Fibronectin and α-SMA to different extents, with a dose-dependent trend observed (*: p<0.05 vs. Control; **: p<0.01 vs. Control; #: p<0.05 vs. HG; ##: p<0.01 vs. HG).

In the present disclosure, the therapeutic effects of LY2922470 and TAK875 on kidney diseases were evaluated using animal models of renal injury and renal failure induced by adenine, as well as in vitro models of renal fibrosis. The experimental results demonstrated that LY2922470 has protective effects on renal function and is effective in treating renal injury, chronic kidney disease, and renal failure. These therapeutic effects are not directly related to the GPR40 target. The anti-fibrotic effect of LY2922470 was confirmed using in vitro models of fibrosis induced by TGF-β1 and high glucose. Therefore, the present disclosure verifies the significant potential of LY2922470 as a therapeutic agent for kidney diseases, highlighting its promising clinical application prospects.

What is claimed is:

1. A method for preventing or treating a kidney disease, the method comprising:
administering to a subject a medicament containing a pharmaceutically effective amount of LY2922470;
wherein the LY2922470 has the following structural formula:

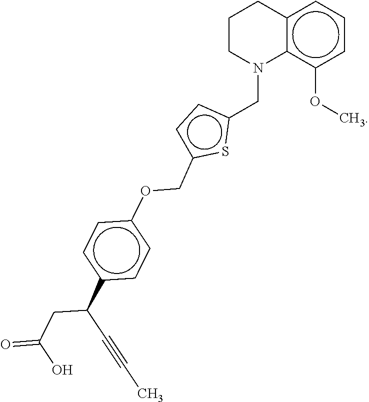

2. The method of claim 1, wherein the kidney disease is renal injury, chronic kidney disease, or renal failure.

3. The method of claim 1, wherein the kidney disease is nephropathy caused by renal fibrosis.

4. The method of claim 1, wherein the kidney disease includes at least one of ischemia-reperfusion (IR)-induced nephropathy, nephrotoxin-induced nephropathy, chemotherapeutic drug-induced nephropathy, hypertension-induced nephropathy, ureteral obstruction-induced nephropathy, and diabetic nephropathy.

5. The method of claim 1, wherein the LY2922470 in the medicament is in a form of a compound or a pharmaceutically acceptable salt thereof.

6. The method of claim 1, wherein the medicament is in an oral or injectable dosage form, and the oral or injectable dosage form includes at least one of a powder, a tablet, a granule, a capsule, an oral liquid, an emulsion, and a suspension.

7. The method of claim 1, wherein the medicament further includes an excipient or an adjunctive medicament for protecting kidneys and blood vessels.

8. The method of claim 1, wherein the medicament further includes a pharmaceutically acceptable carrier, the carrier includes at least one of a diluent, a buffer, a suspending agent, an emulsifying agent, a granulating agent, an encapsulating agent, an excipient, a filler, a binder, a spray agent, a transdermal absorption enhancer, a wetting agent, a disintegrant, an absorption enhancer, a surfactant, a colorant, a flavoring agent, and an adsorbent carrier.
